(19) United States
(12) Patent Application Publication
(10) Pub. No.: US 2010/0152539 A1
(43) Pub. Date: Jun. 17, 2010

(54) POSITIONABLE IMAGING MEDICAL DEVICES

(75) Inventors: Ragae M. Ghabrial, Loveland, OH (US); Michael J. Stokes, Cincinnati, OH (US); Rudolph H. Nobis, Mason, OH (US); Andrew M. Zwolinski, Cincinnati, OH (US); Michael P. Weir, Blanchester, OH (US); David N. Plescia, Cincinnati, OH (US); Christle M. Cunningham, Benson, NC (US); Omar J. Vakharia, Cinicinnati, OH (US); William D. Fox, New Richmond, OH (US); James T. Spivey, Cincinnati, OH (US)

Correspondence Address: K&L Gates LLP, 210 SIXTH AVENUE, PITTSBURGH, PA, 15222-2613, US

(73) Assignee: Ethicon Endo-Surgery, Inc., Cincinnati, OH (US)

(21) Appl. No.: 12/337,340

(22) Filed: Dec. 17, 2008

(51) Int. Cl. A61B 1/00

(52) U.S. Cl. 600/118

(57) ABSTRACT

A positionable imaging device includes a body defining a first end and a second end. The body is configured to be received within an internal body cavity. An imaging device is located at the first end of the body. A releasable fastener is coupled to the body to removably attach the imaging device to tissue within the internal body cavity. A release mechanism is coupled to the releasable fastener to detach the imaging device from the tissue.

### BACKGROUND

The various embodiments relate generally to positionable imaging devices for medical applications. More particularly, the various embodiments relate to positionable imaging devices configured to be received within an internal body cavity, appliers for attaching the imaging devices to body tissue within the internal body cavity, and manipulators for orienting and positioning the imaging device are disclosed.

Minimally invasive surgical procedures, such as endoscopic and laparoscopic procedures, often call for the introduction of medical devices inside a patient's body. For the patient's comfort, the introduction and placement of such devices should be quick, easy, efficient, and reversible. Flexible endoscopes are generally inserted inside the patient through a natural opening such as the mouth, anus, or vagina, although it is more common to use rigid endoscopes for the latter. From the entry point, endoscopes are adapted with steering mechanisms to guide the flexible shaft of the endoscope through the tortuous path of an inner body lumen. Laparoscopes are inserted into the peritoneal cavity through trocars, which are inserted through the abdominal wall via a small—keyhole—incision. Both endoscopes and laparoscopes provide means for viewing the internal portions of a patient's anatomy.

In a conventional laparotomy, a surgical incision made into the abdominal wall to examine internal abdominal organs, the clinician has a direct view of the internal anatomy. In other words, the clinician's view is not coming through an imaging device such as a charge coupled device (CCD) camera. This view of the internal anatomy, often referred to as the “stadium view” or “bird's eye view,” is preferred or desired by many clinicians. Among some of the drawbacks of conventional laparoscopes and endoscopes is the inability to provide the clinician with the same view of the anatomy as provided with a conventional laparotomy. Endoscopes and laparoscopes are available in wide angle or narrow angle varieties. General purpose laparoscopes have longer focal distances than flexible endoscopes, as they are held farther away from the working site (e.g., 6 to 12 inches) than a flexible endoscope held within a bodily lumen (often much less than an inch from the tissue). Some wide angle flexible endoscopes (some near 180 degrees) approach the filed of view of a human. Conventional endoscopes and laparoscopes employ a viewing port at a distal end thereof to transmit images within its field of view to an imaging device such as a CCD camera located within the endoscope so that an operator can view the images of the internal anatomy on a display monitor. In this respect, an endoscope can operate at shorter working distances than a laparoscope. Nevertheless, however, because the imaging device is part of the endoscope, during a procedure, the clinician is required to bring the tip of the endoscope close to the worksite in order to perform the operation. Therefore, the preferred external view of the internal anatomy achievable in open surgical techniques cannot be achieved with conventional endoscopes and laparoscopes.

Introduction of surgical instruments through one or more of the working channels of the endoscope limits the clinician's ability to “triangulate” his or her actions between the viewing port and the surgical tools, especially when all devices are located substantially along a single axis defined by the shaft of the endoscope. Introduction of the surgical tools through various working channels of the endoscope also compromises the flexibility of the endoscope and limits the clinician's ability to navigate and orient the endoscope to obtain a desired image of the internal anatomy. In addition, reaching the worksite with a flexible endoscope involves navigating the endoscope through tortuous internal body lumen paths, making it difficult to end up with the viewing port in the desired rotational orientation when the imaging device is collocated with the endoscope. Thus, the endoscope may not be aligned with a preferred view of the internal anatomy. Correcting the orientation can be very difficult. Finally, the presence of the imaging device and associated wiring takes up valuable space that could be used for more sophisticated and/or larger therapeutic or diagnostic devices.

Accordingly, there is a need for positionable imaging devices appliers therefor. There is also a need for attachment mechanisms for attaching the positionable imaging devices to internal portions of the patient's anatomy to provide a view of the internal anatomy that is decoupled from the orientation of the endoscope.

### FIGURES

The novel features of the embodiments described herein are set forth with particularity in the appended claims. The embodiments, however, both as to organization and methods of operation may be better understood by reference to the following description, taken in conjunction with the accompanying drawings as follows.

FIG. 1 illustrates a schematic view of an imaging device.

FIG. 2 illustrates one embodiment of a positionable imaging device.

FIG. 2A is a magnified view of a pin slidably releasing from a loop when a memory alloy is actuated and transitions from a first state to a second state.

FIG. 3 illustrates one embodiment of a positionable imaging device.

FIG. 4 illustrates one embodiment of a positionable imaging device.

FIG. 5 illustrates one embodiment of a positionable imaging device shown in use in the peritoneal cavity during deployment.

FIG. 6 illustrates one embodiment of the positionable imaging device shown in use in the peritoneal cavity after deployment.

FIG. 7 illustrates a front view of one embodiment of an electromagnet located outside the peritoneal wall of a patient.

FIG. 8 illustrates one embodiment of a positionable imaging device shown in use attached to the peritoneal wall and facing inwardly towards the peritoneal cavity.

FIG. 9A illustrates a second side of a first body portion of the positionable imaging device shown in FIG. 8.

FIG. 9B illustrates a second side of a second body portion of the positionable imaging device shown in FIG. 8.

FIG. 10 is a block diagram illustrating the functional components of a system for operating one embodiment of the positionable imaging device shown in FIG. 8.

FIG. 11 is a functional block diagram of the system shown in FIG. 8 illustrating the signal flows.

FIG. 12 illustrates one embodiment of a positionable imaging device.

FIG. 13 illustrates one embodiment of the positionable imaging device shown in FIG. 12 deployed via a flexible endoscope with a grasper for holding the imaging device during deployment.

FIGS. 14A-B illustrate one embodiment of the positionable imaging device shown in FIGS. 12-13 comprising a plurality of openings formed in a base portion to receive a tissue fastener therethrough.

FIGS. 15A-B illustrate one embodiment of the positionable imaging device shown in FIGS. 12-13 comprising a vacuum chamber formed in a base portion and a fluid port in fluid communication with the vacuum chamber.

FIGS. 16A-B illustrate one embodiment of the positionable imaging device shown in FIGS. 12-13 comprising a plurality of barbs to penetrate internal tissue such as the peritoneal wall are formed on a second side of a base portion.

FIG. 17 illustrates one embodiment of a positionable imaging device.

FIG. 18 illustrates a perspective view of one embodiment of a positionable imaging device attached to the peritoneal wall with one or more fasteners.

FIG. 19 is a partial cross-sectional view of one embodiment of the positionable imaging device shown in FIG. 18 coupled to a deployment mechanism.

FIG. 20 is a partial cross-sectional view of one embodiment of the positionable imaging device shown in FIG. 18 attached to the peritoneal wall shown in the deployment stage.

FIG. 21 illustrates a partial cross-sectional view of one embodiment of the positionable imaging device shown in FIG. 18 attached to the peritoneal wall with one or more hooks.

FIG. 22 illustrates one embodiment of the positionable imaging device shown in FIG. 18 rotatably positioned as a result of applying a force in direction “J” on the end of a percutaneous filaments inserted through the peritoneal wall.

### DESCRIPTION

Before explaining the various embodiments of the positionable imaging devices in detail, it should be noted that the embodiments are not limited in their application or use to the details of construction and arrangement of parts illustrated in the accompanying drawings and description. The illustrative embodiments may be positioned or incorporated in other embodiments, variations and modifications thereof, and may be practiced or carried out in various ways. The positionable imaging devices disclosed herein are illustrative only and not meant to limit the scope or application thereof. Furthermore, unless otherwise indicated, the terms and expressions employed herein have been chosen for the purpose of describing the embodiments for the convenience of the reader and are not to limit the scope thereof.

In the following description, like reference characters designate like or corresponding parts throughout the several views. Also, in the following description, it is to be understood that terms such as front, back, inside, outside, top, bottom and the like are words of convenience and are not to be construed as limiting terms. Terminology used herein is not meant to be limiting insofar as devices described herein, or portions thereof, may be attached or utilized in other orientations. The various embodiments will be described in more detail with reference to the drawings.

Various embodiments of positionable imaging devices, e.g., cameras, and elements thereof disclosed herein may be introduced within a patient using minimally invasive surgical techniques (e.g., endoscopically, laparoscopically), conventional open surgical techniques (e.g., laparotomy), or percutaneously. For example, the various embodiments of the positionable imaging devices described herein may be inserted through a trocar, flexible endoscope, overtube, or incision. Minimally invasive techniques provide access to a worksite within an internal body cavity of the patient for diagnostic and treatment procedures to treat tissue, perform a biopsy, or perform surgery. It is essential for the user to guide working tools to precise locations in the workspace, and while non-visual imaging may be employed (e.g., ultrasound, x-ray), simple visual imaging is the current standard, and represents the mental “image” that users have of the anatomy. Therefore, in some instances it may be advantageous to introduce a positionable imaging device into the patient. Accordingly, various embodiments of positionable imaging devices disclosed herein may be used in endoscopic and/or laparoscopic surgical procedures, conventional laparotomies, or any combinations thereof.

In one embodiment, the positionable imaging devices disclosed herein may be introduced through a natural opening of the body such as the mouth, anus, and/or vagina and delivered to the desired internal anatomical site using trans-organ or translumenal surgical procedures. In a natural orifice translumenal endoscopic procedure, such as the procedures developed by Ethicon Endo Surgery, Inc. known in the art as Natural Orifice Translumenal Endoscopic Surgery (NOTES™), the flexible portion of an endoscope is introduced into the patient through one or more natural openings and is guided to the anatomical site using direct line-of-sight, cameras, or other imaging devices formed integrally with the endoscope. Surgical devices used to perform key surgical activities at the worksite, including the various embodiments of the positionable imaging devices disclosed herein, may be introduced through the one or more working channels of the endoscope. Although some embodiments of the positionable imaging device is intended to be used outside a lumen within an internal body cavity of the patient, translumenal techniques may be employed for introducing surgical working tools through an inner body lumen and breaking through the lumen to access extraluminal organs located within the internal body cavity. In one embodiment, the positionable imaging device may be introduced intraluminally in order to navigate to the exit point for use outside the lumen.

As previously discussed, various embodiments of positionable remote imaging devices disclosed herein may be employed in endoscopic, laparoscopic, open surgical procedures, or any combinations thereof. Endoscopy is a minimally invasive surgical procedure vehicle for performing minimally invasive surgery and refers to looking inside the human anatomy for medical reasons. Endoscopy may be performed using an instrument called an endoscope, which may have a rigid shaft, flexible shaft, or a combination thereof. Endoscopy may be used to evaluate the surfaces of organs or to perform internal surgery. The endoscope provides images of surface conditions of the organs including abnormal or diseased tissue such as lesions and other surface conditions, and in some models the endoscope may be adapted and configured for taking biopsies, retrieving foreign objects, and introducing medical instruments to the worksite. Generally this type of visual imaging is referred to as “first surface” imaging. The user sees the first surface rays drawn from the endoscope to the tissue intersect. Ordinarily the user cannot see behind, underneath, or through the tissue. On the other hand, a confocal microscope endoscopes working in visible wavelengths (such as those produced by Pentax, for example) may see somewhat beneath the surface. An ultrasound endoscope (such as those produced by Hitachi, Olympus, for example) sees well below the surface.

Laparoscopic and thoracoscopic surgery are encompassed within the broader field of endoscopy. Laparoscopy and thoracoscopy also are minimally invasive surgical techniques in which operations in the abdomen are performed through small incisions (usually 0.5 cm-1.5 cm), keyholes, as compared to larger incisions or laparotomies, needed in traditional open surgical procedures. Laparoscopic surgery refers to operations performed within the abdominal or pelvic cavities, whereas keyhole surgery operations performed within the thoracic or chest cavity are referred to as thoracoscopic surgery. In a laparoscopic procedure the laparoscope may be inserted through a 5 mm or 10 mm trocar or keyhole to view the operative field. The abdomen is usually insufflated with carbon dioxide gas elevating the abdominal wall above the internal organs like a dome to create a working and viewing space. Carbon dioxide gas is used because it is common to the human body and can be removed by the respiratory system if it is absorbed through tissue.

In various embodiments, the positionable imaging devices described hereinbelow with reference to the specific embodiments may be employed in preoperative patients to screen and diagnose diseases, evaluate tissue without surgery, and to monitor, scan, or otherwise visualize a treatment site inside the patient prior to surgery. The various embodiments of the positionable imaging devices described herein may be employed in surgical therapy to administer sedatives, anesthetics, perform surgical procedures, and to visualize the treatment site or worksite within the patient during surgery. When positioned at the worksite, the positionable imaging devices illuminate and provide images of the internal anatomy to enable the clinician to more accurately diagnose and provide effective treatment. Embodiments of the positionable imaging devices may provide images of the desired tissue during in-vivo treatment procedures used to ablate or destroy live cancerous tissue, tumors, masses, lesions, and other abnormal tissue growths present at the tissue treatment site.

In various embodiments, the positionable imaging devices described hereinbelow with reference to the specific embodiments may comprise an attachment mechanism. The attachment mechanism may be employed to quickly and easily removably attach the imaging device to body tissue within the internal body cavity of the patient. The reversible attachment mechanism enables quick and easy attachment, detachment, positioning, repositioning, and/or removal of the positionable imaging device. The attachment mechanism may be actuated using standard commercially available appliers or may be actuated with custom appliers. The attachment mechanism may be employed to locate the device at a worksite and quickly and easily actuate the attachment mechanism to secure the device to the internal body tissue of the patient.

In various embodiments, the positionable imaging devices described hereinbelow with reference to the specific embodiments may be configured to provide images of the worksite or desired internal anatomy including the lungs, liver, stomach, digestive tract including the small and large intestines and the colon, gall bladder, urinary tract, reproductive tract, intestinal tracts, and/or the peritoneal cavity, for example. Images may be obtained during the deployment process as the positionable imaging device advances through internal body lumen and cavities, and when the device is attached to internal tissue to illuminate and image the operative field and provide a view of the worksite during the surgical or diagnostic procedure.

A key element in endoscopic, laparoscopic, or thoracoscopic surgery is the use of a scope, which may include rigid or flexible lens based systems, that is usually connected to a video camera (single chip or multi chip) or a distal CCD video camera based system that places the video camera optics and electronics at the tip of the scope. Also attached to the proximal end of the scope may be a fiber optic cable system connected to a “cold” light source (halogen or xenon) to illuminate the operative field. Alternatively, illumination may be achieved using a solid-state element, such as a light emitting diode (LED) placed at the distal end of the laparoscope.

In various embodiments, the positionable imaging devices described hereinbelow with reference to the specific embodiments may comprise single or multiple imaging devices to provide a suitable range of image acquisition capabilities. In other embodiments, the positionable imaging devices may comprise a plurality of imaging devices arranged to provide image acquisition capabilities in multiple orientations. In one embodiment, the positionable imaging devices are coupled wirelessly or though wires to an image acquisition system to and display the images on a video monitor outside located outside the patient.

In various embodiments, the imaging device component of the positionable imaging devices described hereinbelow with reference to the specific embodiments may be configured to convert images into electrical signals, which can be transmitted to a remote receiver where the signals are converted back into viewable images and displayed on a video monitor. The signals may be transmitted outside the patient either wirelessly or through electrical conductors placed percutaneously, through the same access path as the translumenal endoscopic access device, or though any suitable percutaneous, lumenal, or translumenal path. In wireless applications, the imaging device may comprise either a transmitter or a transceiver (e.g., transmitter/receiver) and an antenna.

In various embodiments, the imaging device component of the positionable imaging devices described hereinbelow with reference to the specific embodiments may be energized by on-board energy sources, such as one or more batteries. In other embodiments, the imaging devices may be energized by remote energy sources coupled to the imaging device either wirelessly using wireless energy transfer techniques or through electrical conductors, which may introduced percutaneously, along the same path as the translumenal endoscopic access device, or any suitable path.

In various embodiments, the positionable imaging devices described hereinbelow with reference to the specific embodiments may employ a CCD or complementary metal oxide semiconductor (CMOS) camera. As used herein, the term “camera” is intended to cover any imaging device comprising image sensors suitable for capturing light and converting images to electrical signals that can be stored in electronic storage media or transmitted to external devices for displaying the images on video monitors. The images may include still photographs or a sequence of images forming a moving picture (e.g., movies or videos). Optical systems comprising one or more lenses may be optically coupled to the one or more image sensors, similar to those employed in digital cameras and other electronic imaging devices, to convert an optical image to an electric signal. The image sensor may comprise one or more arrays of CCD or CMOS devices such as active-pixel sensors. A large area image sensor may be used to provide image quality equivalent to that obtainable with standard laparoscopes. A typical image sensor may comprise a sensor array with an image input area of approximately 10 mm diameter. The imaging device also may comprise elements for orienting, panning, zooming, and/or focusing optical system to provide an optimal viewing angle of the target anatomy in a desired orientation.

The imaging device is coupled to a circuit comprising any necessary electronic components or elements for processing, storing, and/or transmitting the images received by the image sensor. The images may be processed by any suitable digital or analog signal processing circuits and/or techniques implemented in logic, software, or firmware. Furthermore, the images may be stored in electronic storage media such as, for example, memory devices. The circuits may be coupled by one or more connectors. It will be appreciated by those skilled in the art that a single circuit or multiple circuits may be employed to process, store, and transmit the images without limiting the scope of the illustrated embodiments.

The circuits, image sensors, batteries, illumination sources, transmitters, transceivers, antennas, and/or any other electrical component, may be disposed on a variety of substrates such as a printed circuit board and/or ceramic substrate and may be connected by one or more connectors. A port may be provided to receive electrical conductors for carrying image signals or for carrying electric power to the imaging device. The electrical conductors may be removably connected to one or more connectors coupled to a circuit board.

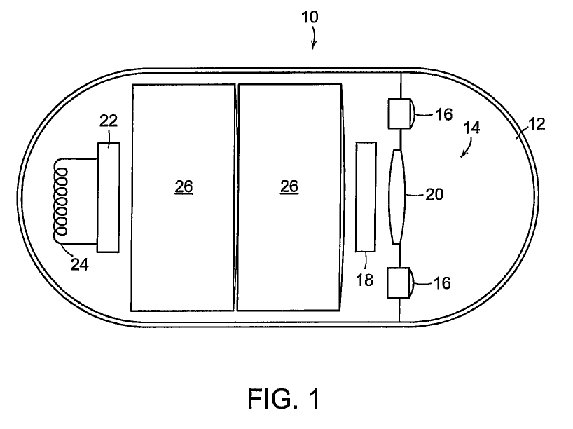

FIG. 1 illustrates a schematic view of an imaging device 10. The imaging device 10 may be employed for viewing inside body cavities and for transmitting at least video data. FIG. 1 illustrates the imaging device 10 and its components. The imaging device 10 typically comprises an optical window 12 and an imaging system 14 for obtaining images from inside a body cavity, such as the gastrointestinal (GI) tract. The imaging system 14 comprises an illumination source 16, such as a white LED, an imaging camera 18 (e.g., CCD, CMOS), which detects the images, and an optical system 20 which focuses the images onto the imaging camera 18. The illumination source 16 illuminates the inner portions of the body cavity through an optical window 12. The imaging device 10 further includes a transmitter 22 and an antenna 24 for transmitting the video signal of the imaging camera 18, and an energy source 26 that provides power to the electrical elements of the device 10.

The energy source 26 may comprise one or more batteries, such as a silver oxide battery. The energy source 26 may be an on-board energy source located within a housing or body of the imaging device 10, such as a battery or may be a remote energy source located outside the housing or body of the imaging device 10. Percutaneous electrical conductors or electrical conductors introduced along a translumenal endoscopic access device may be used to supply the imaging device 10 with power from a remote energy source. In other embodiments, the imaging device 10 may be powered by remote energy sources using wireless energy transfer techniques such as induction or resonant induction. Wireless energy transfer or wireless power transmission is the process of transmitting electrical energy from an energy source to an electrical load, without interconnecting wires. An electrical transformer is the simplest instance of wireless energy transfer. The primary and secondary circuits of a transformer are not directly connected. The transfer of energy takes place by electromagnetic coupling through a process known as mutual induction. Wireless power transfer technology using RF energy is produced by Powercast, Inc. The Powercast system achieves a maximum output of 6 volts for a little over one meter. Other low-power wireless power technology has been proposed such as described in U.S. Pat. No. 6,967,462.

It will be appreciated that a plurality of CMOS imaging cameras may be used in the imaging device 10 and system. Each CMOS imaging camera may include its own optical system and either one or more illumination sources, in accordance with specific requirements of the device or system.

Images obtained by the imaging camera 18 are transmitted to a receiving system (not shown), which may also include a data processing unit. The receiving system and data processing unit are typically located outside a patient. The images may be processed using any suitable digital or analog signal processing circuits and/or techniques. Furthermore, the images may be stored in electronic storage media such as, for example, memory devices. The images may be transmitted wirelessly to external devices for storing, displaying, or further processing the images in real-time. In various embodiments, the images may be transmitted over endoscopic, laparoscopic, or transcutaneous wires inserted within the internal body cavity where the imaging device 10 is located.

The imaging device 10 may be of any shape suitable for being inserted into an internal body cavity. Furthermore, the imaging device 10 may be attached or affixed on to an instrument that is inserted into body lumens and cavities, such as on an endoscope, laparoscope, stent, needle, and catheter. Thus, the imaging device 10 may be introduced into the internal body cavity using an endoscopic device or by open surgical techniques.

A suitable imaging camera 18 is, for example, a “camera on a chip” type CMOS imager with integrated active pixel and post processing circuitry. The single chip camera can provide either black and white or color signals. The imaging camera 18 may be designed such that it is less sensitive to light in the red spectrum than known CMOS cameras. The imaging camera 18 may comprise one or more CCD arrays or CMOS devices such as active-pixel sensors. The imaging camera 18 captures light and converts it into electrical signals. A large area image sensor may be used to provide a substantially high quality image equivalent to that obtainable which may be obtained with standard laparoscopes, for example. In one embodiment, the imaging camera 18 may comprise a sensor array having approximately a 10 mm diameter image input area. In other embodiments, motors may be employed for orienting, panning, zooming, and/or focusing the imaging camera 18 and providing an optimal viewing angle of the target anatomy in a desired orientation.

The optical system 20 comprises at least one lens and optionally mirrors and/or prisms for collecting and collimating remitted light on to the pixels of the imaging camera 18. Typically, the optical system comprises an aspherical focusing lens. A suitable lens may be designed in accordance with specific object plane, distortion and resolution parameters.

The illumination source 16 transmits light to the walls of the internal body cavity via the optical window 12. The lens of the optical system 20 then focuses remittent light onto the pixels of the imaging camera 18.

A single or plurality of illumination sources or a specific integrated illumination source may be used and positioned in accordance with specific imaging requirements, such as to avoid stray light. Also, the optical window 12 may be positioned and shaped according to the device shape and according to specific imaging requirements. For example, optimized imaging conditions can be obtained when optical window 12 is formed to define an ellipsoid shaped dome and the imaging camera 18 and illumination sources 16 are positioned in the proximity of the focal plane of the shape defined by the optical dome.

The in-vivo sites imaged are usually very close to the imager. It is therefore possible to satisfy the illumination requirements of the imaging process utilizing solid state illumination sources, such as one or more LEDs.

In one embodiment, the illumination source is a white LED. The white light emitted from the white LED has a small faction of red light and even smaller fraction of infrared (IR) light. Hence, a white LED is beneficial for use with silicone based image sensors (such as CMOS imaging cameras) because of the silicone sensitivity to red and IR light. In a system which includes the imaging camera 18 with its reduced sensitivity to light in the red spectrum and a white LED illumination source, no IR reject filters (photopic filters) are needed. One or more illumination sources 16 may be located on either ends of the body to illuminate the site to be imaged. The illumination source 16 may comprise one or more light sources such as LEDs. In one embodiment, the illumination source 16 may comprise a single LED or a combination of LEDs to produce light of a desired spectrum. In one embodiment, the illumination source 16 may be coupled to motors for orienting, panning, zooming, and/or focusing the illumination source 16 to provide optimal illumination of the target site.

A suitable transmitter may comprise a modulator which receives the video signal (either digital or analog) from the imaging camera 18, a radio frequency (RF) amplifier, an impedance matcher and an antenna. In wireless applications, the imaging device 10 may comprise a transceiver (e.g., transmitter/receiver) to transmit the video signal from the imaging camera 24 and to receive command signals for operating aspects of the imaging device 10 remotely.

The imaging device 10 can additionally include sensor elements for measuring pH, temperature, pressure. These sensor elements, some of which are described in the prior art, may be any element suitable for measuring conditions prevailing in the body cavity (for example, the digestive system) and that are capable of being appended to or included in the device.

One or more substrates (e.g., printed circuit boards, ceramic) may be used to mechanically support and electrically connect any of the electronic components associated with the imaging device 10 using conductive pathways, or traces. The substrate may be a rigid or flexible printed circuit board, ceramic, or may be formed of other suitable materials, and may be interconnected by one or more connectors.

Additional details of the imaging device 10 may be similar to those described in U.S. Pat. Nos. 5,604,531 and 7,009,634, each of which is incorporated herein by reference in its entirety.

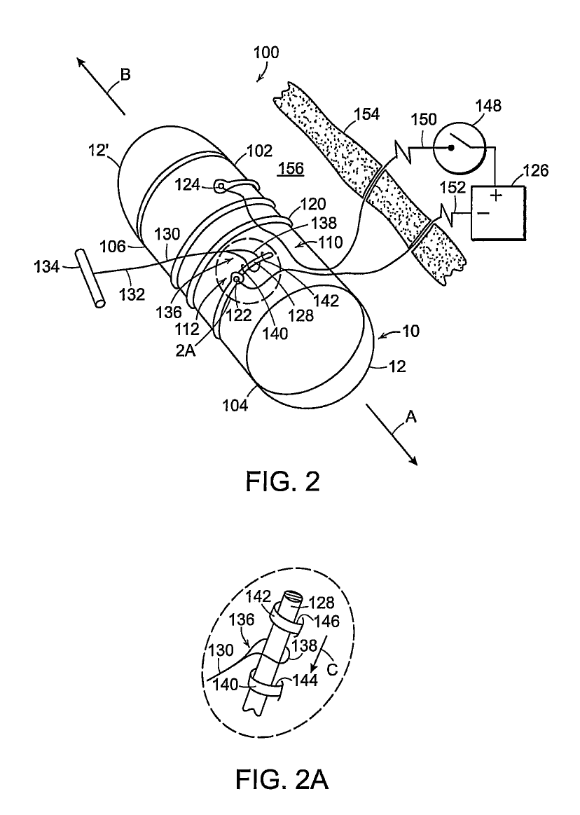

FIG. 2 illustrates one embodiment of a positionable imaging device 100. In one embodiment, the positionable imaging device 100 comprises a body 102 defining a first end 104 and a second end 106. The body 102 is configured to be received within an internal body cavity 156 of the patient such as the peritoneal cavity. The body 102 may be shaped according to specific positioning and imaging requirements and, in the illustrated embodiment the body 102 has a substantially cylindrical configuration. A releasable fastener 110 is coupled to the body 102 to removably attach the positionable imaging device 100 to tissue within the internal body cavity of the patient. A release mechanism 112 is coupled to the releasable fastener 110 to detach the positionable imaging device 100 from the tissue.

In one embodiment, the positionable imaging device 100 comprises one embodiment of the imaging device 10 described in FIG. 1 for viewing inside body cavities and for transmitting at least video data. The positionable imaging device 100 may be employed for viewing inside body cavities in direction “A” through the optical window 12 located at the first end 104 of the body 102. In one embodiment, the positionable imaging device 100 may comprise another optical window 12′ located at the second end 106 of the body 102 for viewing inside body cavities in direction “B.” The first and second optical windows 12, 12′ each may have a hemispherical, ellipsoid shaped dome or rounded configuration. In various embodiments, the positionable imaging device 100 may comprise one or more imaging devices 10 and optical windows 12, 12′ such that the viewing direction “A” or “B” may be selectable by the user. Thus, when the positionable imaging device 100 is deployed within the internal body cavity of the patient and attached to the patient's anatomy, the imaging system 10 can acquire images in either/or both direction “A” or “B.”

In one embodiment, the positionable imaging device 100 comprises an elongate memory alloy 120 having a first end 122 and second end 124. The first end 122 of the memory alloy 120 is anchored, e.g., fixedly attached, to the body 102. The second end 124 of the memory alloy 120 is removably attached to the body 102. The memory alloy 120 is actuatable from a first state to a second state by an energy source 126 coupled between the first and second ends 122, 124 of the memory alloy 120. A switch 148 is coupled between the first end 122 of the memory alloy 120 and the energy source 126. As shown, first and second electrical conductors 150, 152 are introduced transcutaneously through the patient's skin and through an internal body wall 154, such as the peritoneal cavity. A pin 128 may be coupled to the first end 122 of the memory alloy 120 to removably couple the memory alloy 120 to the body 102. A first end 132 of a length of suture 130 is coupled to a tissue anchor 134. A second end 136 of the length of suture 130 defines a loop 138 that is removably coupled to the pin 128. The loop 138 at the second end 136 of the suture 130 may be threaded to the pin 128. In one embodiment, the tissue anchor 134 may be a T-tag, which may be applied using a T-tag tissue apposition system (TAS), for example.

As shown in FIG. 2A, when the memory alloy 120 is actuated, the memory alloy 120 transitions from the first state to the second state and the pin 128 slidably releases from the loop 138 in direction “C” and is disconnected from the body 102. In one embodiment, the body 102 comprises first and second axially aligned projections 140, 142 defining corresponding first and second openings 144, 146 to slidably receive the pin 128 therethrough. In one embodiment, the memory alloy 120 may be formed of NITINOL® wire having a first length in the first state and having a second, shorter, length in the second state. Thus, when a voltage is applied to the NITINOL® wire, the wire decreases in length and the pin 128 slidably moves in the direction indicated by arrow “C” to release the loop 130 and thus release the body 102 of the positionable imaging device 100 from the tissue anchor 134.

In one embodiment, the positionable imaging device 100 may be deployed using minimally invasive surgical procedures (e.g., endoscopic, laparoscopic, thoracoscopic, or any combination thereof). In the illustrated embodiment, the positionable imaging device 100 is configured to be attached within the internal body cavity 156 of the patient. When the positionable imaging device 100 is positioned at the desired treatment site within the internal body cavity, the positionable imaging device 100 is anchored to tissue proximal the treatment site by the tissue anchor 134. In the anchored position, the positionable imaging device 100 is employed to monitor the treatment site during surgery and to monitor healing and tissue response to therapy over time after the surgery. The positionable imaging device 100 is remotely released by actuating the memory alloy 120 and allowed to pass through the GI tract when the treatment is complete and monitoring is no longer required. In one embodiment, the positionable imaging device 100 also may be configured for time delayed release of one or more therapeutical substances into the patient.

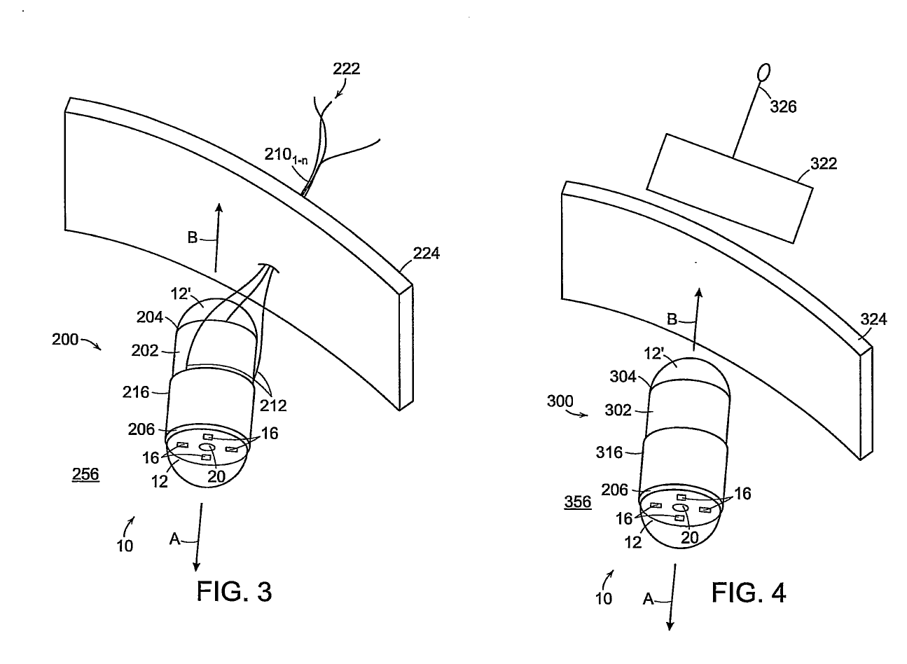

FIG. 3 illustrates one embodiment of a positionable imaging device 200. In one embodiment, the positionable imaging device 200 comprises a body 202 defining a first end 204 and a second end 206. The body 202 is configured to be received within an internal body cavity 256 of the patient such as the peritoneal cavity. The body 202 may be shaped according to specific positioning and imaging requirements and, in the illustrated embodiment, the body 202 has a substantially cylindrical configuration. A plurality of percutaneous filaments 210₁₋ₙ, where n is any suitable positive integer, each have a first end 212 fixedly attached to the body 202. The plurality of percutaneous filaments 210a-n may be circumferentially positioned about the outer portion of the cylindrical body 202, for example. A collar 216 is positioned over the body 202 and fixedly attached thereto. The plurality of percutaneous filaments 210a-n may be circumferentially fixedly attached to the collar 216. Free ends 222 of the plurality of percutaneous filaments 210a-n are percutaneously inserted through an internal body wall 224, e.g., the peritoneal wall, and are used to manipulate the body 202 of the positionable imaging device 200 to position the positionable imaging device 200 from outside the internal body wall 224.

In one embodiment, the positionable imaging device 200 comprises one embodiment of the imaging device 10 described in FIG. 1 for viewing inside body cavities and for transmitting at least video data. The positionable imaging device 200 may be employed for viewing inside body cavities in direction “A” through the optical window 12 located at the first end 204 of the body 202. In one embodiment, the positionable imaging device 200 may comprise another optical window 12′ located at the second end 206 of the body 202 for viewing inside body cavities in direction “B.” The first and second optical windows 12, 12′ each may have a hemispherical, ellipsoid shaped dome or rounded configuration. In various embodiments, the positionable imaging device 200 may comprise one or more imaging devices 10 and optical windows 12, 12′ such that the viewing direction “A” or “B” may be selectable by the user. Thus, when the positionable imaging device 200 is deployed within the internal body cavity 256 of the patient and attached to the patient's anatomy, the imaging system 10 can acquire images in either/or both direction “A” or “B.”

When the positionable imaging device 200 is deployed, the free ends 222 of the plurality of percutaneous filaments 210₁₋ₙ may be inserted through the internal body wall 224 to the outside of the patient's body. The free ends 222 of the plurality of percutaneous filaments 210a-n may be accessed outside the body through the internal body cavity 256 to independently manipulate and remotely orient and rotate the body 202 to position either one of the optical windows 12, 12′ at a desired viewing angle to visualize the desired anatomy from outside the patient. In one embodiment, the free ends 222 of the plurality of percutaneous filaments 210₁₋ₙ may be coupled to an ergonomic interface (not shown) to assist in the manipulation. In one embodiment, the plurality of percutaneous filaments 210₁₋ₙ may be formed with a degree of stiffness to adequately control and maintain the viewing position.

The positionable imaging device 200 may be deployed inside the internal body cavity 256 of the patient such as the peritoneal cavity using well known minimally invasive procedures used in transgastric, transcolonic, or laparoscopic surgery. In other methods of deployment, the positionable imaging device 200 may be deployed inside the internal body cavity 256 using translumenal access techniques, such as NOTES™, for example, or traditional laparotomies.

FIG. 4 illustrates one embodiment of a positionable imaging device 300. In one embodiment, the positionable imaging device 300 comprises a body 302 defining a first end 304 and a second end 306. The body 302 is configured to be received within an internal body cavity 356 of the patient such as the peritoneal cavity. The body 302 may be shaped according to specific positioning and imaging requirements and, in the illustrated embodiment the body 302 has a substantially cylindrical configuration. A magnetic element 316 is circumferentially positioned over the body 302 and fixedly attached thereto.

In one embodiment, the positionable imaging device 300 comprises one embodiment of the imaging device 10 described in FIG. 1 for viewing inside body cavities and for transmitting at least video data. The positionable imaging device 300 may be employed for viewing inside body cavities in direction “A” through the optical window 12 located at the first end 304 of the body 302. In one embodiment, the positionable imaging device 300 may comprise another optical window 12′ located at the second end 306 of the body 302 for viewing inside body cavities in direction “B.” The first and second optical windows 12, 12′ each may have a hemispherical, ellipsoid shaped dome or rounded configuration. In various embodiments, the positionable imaging device 300 may comprise one or more imaging devices 10 and optical windows 12, 12′ such that the viewing direction “A” or “B” may be selectable by the user. Thus, when the positionable imaging device 300 is deployed within the internal body cavity 356 of the patient, the imaging system 10 can acquire images in either/or both direction “A” or “B.”

In one embodiment, the positionable imaging device 300 interfaces with a magnetic interface 322, such as a manipulatable control rod or joystick, located outside an internal body wall 324 to coact with the magnetic element 316 to remotely position the optical element at a desired viewing angle and rotate the body 302 of the positionable imaging device 300 from outside the patient. The magnetic element 316, e.g., collar, is circumferentially positioned and fixedly attached to the body 302. The magnetic element 316 is configured to coact with the magnetic interface 322 located outside the patient's internal body wall 324 (e.g., peritoneal or abdominal wall) to remotely position the first or second optical element 308, 314 at a desired viewing angle and rotate the positionable imaging device 300 from outside the patient by manipulating the magnetic interface 322. In one embodiment, the magnetic interface 322 may comprises a control rod 326 implemented as a joystick to assist the manipulation of the positionable imaging device 300.

The positionable imaging device 300 may be deployed inside the peritoneal cavity 356 of the patient using well known minimally invasive procedures used during transgastric, transcolonic, or laparoscopic surgery. In one embodiment, the positionable imaging device 300 may be deployed inside the peritoneal cavity using translumenal access techniques, such as NOTES™, for example, or traditional laparotomies.

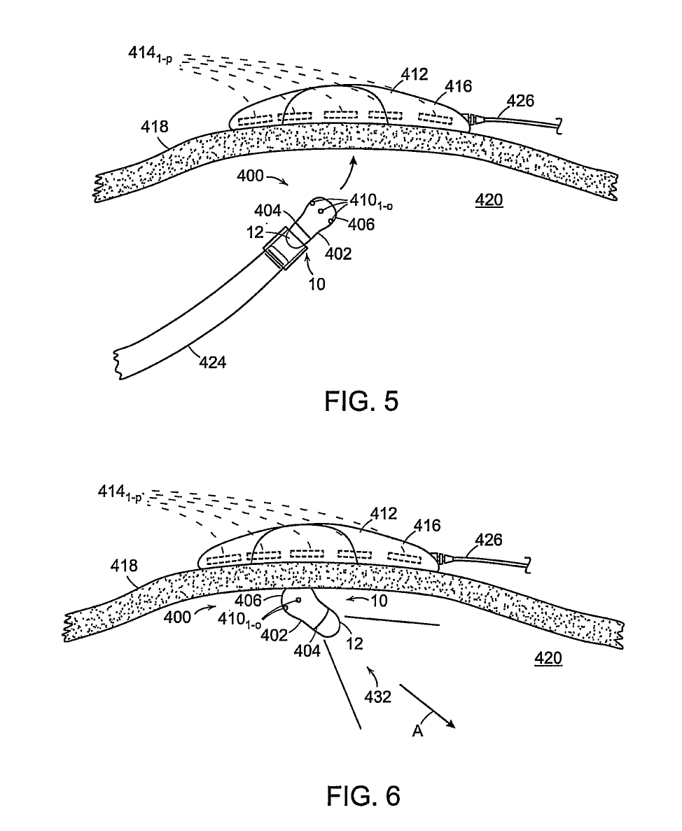

FIG. 5 illustrates one embodiment of a positionable imaging device 400 shown in use in the internal body cavity 420 during deployment. In one embodiment, the positionable imaging device 400 comprises a body 402 defining a first end 404 and a second end 406. The body 402 is configured to be received within the internal body cavity 420 such as the peritoneal cavity. The body 402 may be shaped according to specific positioning and imaging requirements and, in the illustrated embodiment the body 402 has a substantially cylindrical configuration. At least one magnet 410 is located on an external surface of the body 402. In one embodiment, the at least one magnet 410 is located on the second end 406 of the body 402. In one embodiment, a plurality of magnets 410₁₋ₒ, where o is any suitable positive integer, may be located on the second end 406 of the body 402. The positionable imaging device 400 may be positioned or deployed within the internal body cavity 420 with a deployment device 424, such as, for example, a conventional endoscope or a particularly configured endoscopic device. Other suitable deployment devices may be employed. An electromagnet 412 comprising a plurality of electromagnetic elements 414₁₋ₚ is located outside the internal body wall 418 to interact with the plurality of magnets 410₁₋ₒ and control the position of the positionable imaging device 400.

In one embodiment, the positionable imaging device 400 comprises one embodiment of the imaging device 10 described in FIG. 1 for viewing inside body cavities and for transmitting at least video data. The positionable imaging device 400 may be employed for viewing inside body cavities in direction “A” through the optical window 12 located at the first end 404 of the body 402. In one embodiment, the positionable imaging device 100 may comprise another optical window located along the body 102 for viewing inside body cavities. The first and second optical windows 12, 12′ each may have a hemispherical, ellipsoid shaped dome or rounded configuration.

FIG. 6 illustrates one embodiment of the positionable imaging device 400 shown in use in the internal body cavity 420 such as the peritoneal cavity after deployment. In one embodiment, the at least one magnet 410 is configured to coact with the electromagnet 412 comprising the plurality of electromagnetic elements 414₁₋ₚ, where p is any suitable positive integer. From outside the patient, the electromagnet 412 is employed to remotely position and rotate the positionable imaging device 400 by selectively energizing one or more of the plurality of the electromagnetic elements 414₁₋ₚ to locate the positionable imaging device 400 at a desired viewing angle, thus obtaining a desired field of view 432 of the operative of diagnostic field in direction “A.” The field of view 432, viewing angle, and rotation of the positionable imaging device 400 may be manipulated by selectively turning “on” and “off” the plurality of electromagnetic elements 414₁₋ₚ and generating magnetic fields of a desired polarity at different locations to attract or repel one or more of the plurality of magnets 410₁₋ₒ.

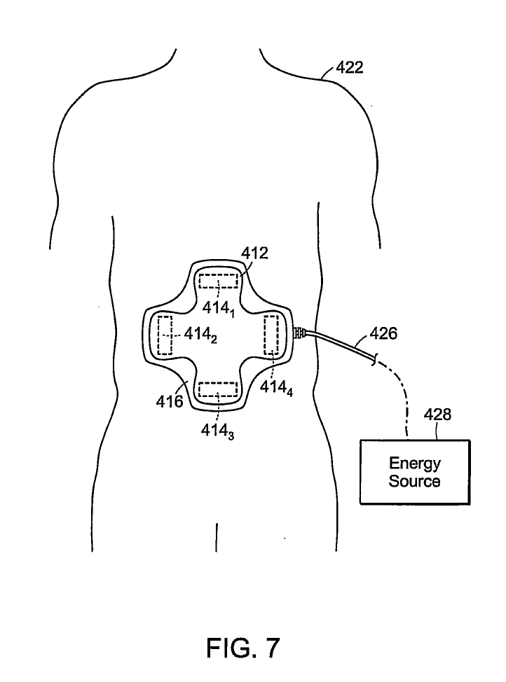

FIG. 7 illustrates a front view of one embodiment of the electromagnet 412 located outside the internal body wall 418 of a patient 422. The electromagnet 412 and the plurality of electromagnetic elements 414₁₋ₚ are contained within a housing 416. In the embodiment illustrated in FIG. 7, the electromagnet 412 comprises four electromagnetic elements 414₁₋₄. The electromagnet 412 is coupled to an energy source 428 via a cable 426. With reference now to FIGS. 4-6, the electromagnet 412 is located outside the internal body wall 418 of the patient 422. Electricity from the energy source 428 is applied to the plurality of electromagnetic elements 414₁₋ₚ to generate magnetic fields of suitable polarity that interact with the magnets 410₁₋ₒ located on the body 402 of the positionable imaging device 400. Thus, the electromagnetic elements 414₁₋ₚ may coact with the magnets 410₁₋ₒ to remotely position or rotate the positionable imaging device 400 at a desired viewing angle and field of view 432 from outside the patient 422. This may be accomplished by selectively energizing the electromagnetic elements 414₁₋ₚ to either attract or repel the magnets 410₁₋ₒ in a coordinated manner to remotely position or rotate the optical element 408.

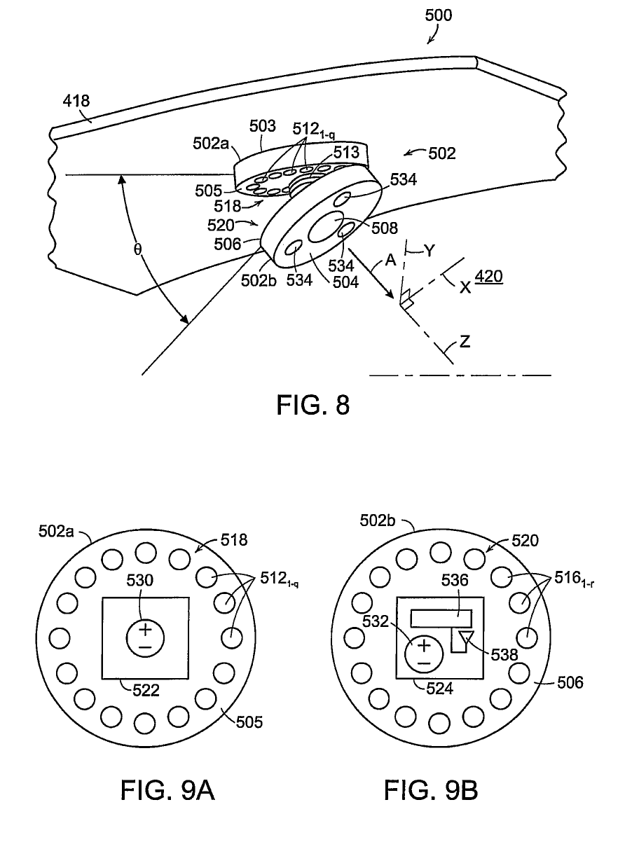

FIG. 8 illustrates one embodiment of a positionable imaging device 500 shown in use attached to the internal body wall 418 and facing inwardly towards the peritoneal cavity 420 in direction “A.” In one embodiment, the positionable imaging device 500 comprises a body 502 defining a first side 504 and a second side 506. The body 502 is configured to be received within an internal body cavity of the patient such as the peritoneal cavity. A magnetic element 516₁₋ᵣ (FIG. 9B) is located on the second side 506 of the body 502. One or more illumination sources 534 are located on the first side of the body 502 arranged to illuminate the operative or diagnostic field in direction “A.” An imaging device 508, similar to the imaging device 10 described in FIG. 1 for viewing inside body cavities and for transmitting at least video data, is located in the center of the body 502 or may be offset from the center to provide an offset field of view. Those skilled in the art will appreciate that the imaging device 508 may comprise the same components, or equivalents, as the imaging device 10 described in FIG. 1, thus, for succinctness, the specific features will not be described.

In the illustrated embodiment, the positionable imaging device 500 comprises a fixable first body portion 502a having a first side 503 configured to attach to internal tissue, such as the internal body wall 418. The first body portion 502a may comprise curved needles, sutures, suction, or adhesives to anchor the first body portion 502a, and hence the positionable imaging device 500, to the peritoneal wall. The first body portion 502a comprises a first plurality of individually controllable electromagnetic elements 512_{1-q}, where q is any suitable positive integer, arranged in a predetermined pattern on a second side 505 (FIG. 9A). A positionable second body portion 502b is coupled to the first body portion 502a by a coupling member 513. The second body portion 502b comprises the imaging device 508 and the illumination sources 534. As previously discussed, the imaging device 508 may be located in the center of the second body portion 502b or may offset from the center to provide an offset viewpoint, similar to an angled laparoscope as compared with a straight or 0 degree laparoscope. The offset imaging device 508 may be favored for more complex procedures because it provides an additional degree of freedom for viewing the scene. In this case the available offset may be limited to the radius of the disk, although an extensible arm could be imagined to carry it further. The second body portion 502b is movable relative to the first body portion 502b. The second body portion 502b comprises a second plurality of individually controllable electromagnetic elements 516₁₋ᵣ (FIG. 9B) where r is any suitable positive integer, arranged in the predetermined pattern. The first and second plurality of electromagnetic elements 514_{1-q}, 516₁₋ᵣ, are remotely coupled to a controller to remotely position the second body portion 502b, and hence, the imaging device 508 at a desired viewing angle and rotate the second body portion 502b from outside the patient by selectively energizing one or more of the plurality of the electromagnetic elements 514_{1-q}, 516₁₋ᵣ. In one embodiment, the number of individually controllable electromagnetic elements 514_{1-q}, 516₁₋ᵣ in the first and second body portions 502a, b may be equal such that q=r. In the illustrated embodiment, the first plurality of individually controllable electromagnetic elements 514_{1-q} is arranged in a first circular array 518 and the second plurality of individually controllable electromagnetic elements 516₁₋ᵣ is arranged in a second circular array 520 (FIG. 9B).

In one embodiment, the coupling member 513 connects the first and second body portions 502a, b at a center point thereof and defines a pivot point therebetween. In one embodiment, the coupling member 513 may be a torsion spring. In other embodiments, the coupling member 513 may be a filament, suture, ball-and-socket arrangement, universal joint, or cross-and-yoke arrangement, for example. The coupling member 513 holds the first and second body portions 502a, b together. Various degrees of constraint and freedom between the first and second body portions 502a, b will now be discussed in terms of orientation location and angle. To define the orientation of an object in three-dimensions, three axes are required for determining location and three axes are required for determining rotation. The origin of location, the direction of location reference axes, and axes about which rotations are made may be selected by definition. The viewing direction along “A,” e.g., the direction where the imaging device 508 is pointed may be defined as the line of sight. Considering that both the first and second body portions 502a, b have separate coordinate systems, for embodiments where the first body portion 502a is stationary or affixed to the internal body wall 418, the second body portion 502b defines a “central” axis Z that is perpendicular to its circular face and passes through its center. Two perpendicular axes lying in the innermost circular face of the first and second body portions 502a, b completes the triad. Following convention, we can define the central axis Z as the “roll” axis and the other two axes X and Y as the “pitch” axis and the “yaw” axis, respectively, for the second body portion 502b. The origin of location may be defined as the center of the innermost faces of the first and second body portions 502a, b. The previously defined pitch X and yaw Y axes may be used for location, with the distance along these axes measured from the origin. Accordingly, the coupling member 513 may be selected to exploit the various degrees of constraint and freedom between the first and second body portions 502a, b. A spring may be combined with any of the embodiments of the coupling member 513 to provide a restoring force in any otherwise free axis. Although a torsion spring is shown in the embodiment illustrated in FIG. 8, other forms of springs may be employed without limitation. Embodiments employing a spring member have a common aspect in that motion in any axis X, Y, Z may be constrained by the stress induced in the spring or some mechanical limit not previously encountered such as wire wrap-up, edge contact, or other designed-in feature, for example.

TABLE 1 shows a summary of angular and location constraints for a spring, ball-and-socket, and cross-and-yoke embodiments of the coupling member 513.

TABLE 1

| | ANGULAR | LOCATION | |
|---|---|---|---|
| NAME | CONSTRAINTS | CONSTRAINT | REMARKS |
| Spring | Stress-limited in | Stress-limited | Helical, leaf, post; |
| | pitch, roll, yaw | in x, y, z | provides restoring force |
| Ball-and- | None | Complete | Presuming low friction |
| socket | | | joint |
| Cross-and- | Complete in roll; | Complete | Classical Hookes joint |
| yoke | none in pitch and | | |
| | yaw | | |

FIG. 9A illustrates the second side 505 of the first body portion 502a and FIG. 9B illustrates the second side 506 of the second body portion 502b of the positionable imaging device 500 shown in FIG. 8. The coupling member 513 is omitted for clarity. As shown schematically in FIGS. 9A, B, a first control circuit 522 may be located in the first body portion 502a to control the activation of the first plurality of individually controllable electromagnetic elements 514_{1-q}. A second control circuit 524 may be located in the second body portion 502b to control the activation of the second plurality of individually controllable electromagnetic elements 516₁₋ᵣ. A first battery 530 may be located in the first body portion 502a to supply electrical power to the first control circuit 522 and/or the first plurality of individually controllable electromagnetic elements 514_{1-q}. A second battery 532 may be located in the second body portion 502b to supply electrical power to the second control circuit 524 and/or the second plurality of individually controllable electromagnetic elements 516₁₋ᵣ. An antenna 538 and a suitable transceiver 536 may be included for wireless transmission and reception of information including, without limitation, for example, receiving power from a remote energy source, receiving control signals or commands for positioning and orienting the positionable imaging device 500, or for transmitting imaging data from the positionable imaging device 500 to external consoles, user output devices, a receiving system, which may also include a data processing unit (not shown).

The imaging device 508 may be located on the fixed first body portion 502a, the movable second body portion 502b, or both the fixed first body portion 502a and the movable second body portion 502b. The imaging device 508 may have orienting, panning, zooming, and/or focusing capabilities as previously described in FIG. 1 with respect to the imaging device 10.

With reference still to FIGS. 8 and 9A, 9B, in use the positionable imaging device 500 may be delivered to the peritoneal cavity 420 with an endoscope through an access overtube. When located in the peritoneal cavity 420, the first body portion 502a may be attached or anchored to the internal body wall 418 with one or more anchors such as curved needles, suture, suction, or adhesives, for example. When in position, the first and second body portions 502a, b coact to orient and position the imaging device 508 mounted to the second body portion 502b. In the illustrated embodiment, the coupling member 513 connects the first and second body portions 502a, b at their center point. The individual electromagnetic elements 514_{1-q}, 516₁₋ᵣ, arranged in the circular arrays 518, 520, can be selectively turned “on” or “off” by the corresponding first and second control circuits 522, 524 circuits contained within the corresponding first and second body portions 502a, b. It will be appreciated by those skilled in the art that the control circuits 522, 524 may be located remotely, e.g., outside the patient's body, to control the individual electromagnetic elements 514_{1-q}, 516₁₋ᵣ remotely. The electromagnetic elements 514₁, 516₁ may be operated such that the first and second body portions 502a, b are attracted to each other at one point and repelled at other points, as shown in FIG. 8, for example, in order to position the imaging device 508 in a desired orientation. By activating and deactivating the electromagnetic elements 514₂, 516₂ in sequence, the second body portion 502b can be made to rotate, for example. By changing the coupling coefficient of the coupling member 513 between the first and second body portions 502a, b the relative angle θ between the first and second body portions 502a, b can be changed relative to the strength of magnetic attraction and repulsion between the individual electromagnetic elements 514_{1-q}, 516₁₋ᵣ. In the illustrated embodiment, the coupling member 513 is a spring, thus the relative angle θ between the first and second body portions 502a, b can be changed by changing the stiffness or length of the spring.

When the first body portion 502a is attached to the internal body wall 418 and the second body portion 502b is free to rotate, the line of sight of the imaging device 508 may be oriented in various directions by controlling the energizing sequence of the electromagnetic elements 514_{1-q}, 516₁₋ᵣ. By varying the relative angle θ between the first and second body portions 502a, b, the imaging device 508 may be pointed in various directions.

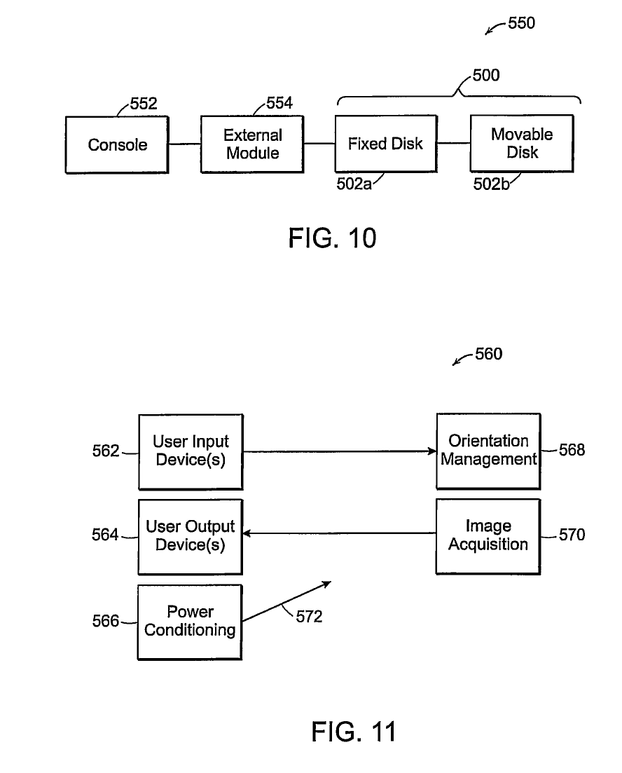

FIG. 10 is a block diagram illustrating the functional components of a system 550 for operating one embodiment of the positionable imaging device 500. The system 550 comprises a console 552, located outside the patient, for displaying the images transmitted by the positionable imaging device 500 and an external module 554 for controlling the positioning of the positionable imaging device 500 from outside the patient. FIG. 11 is a functional block diagram 560 of the system 550 illustrated in FIG. 10 illustrating the signal flows.

With reference now to FIGS. 8 and 9A, 9B, 10, and 11, in one embodiment, a user input device 562, located outside the patient, accepts user specifications such as direction of view, field of view, and video processing options for the positionable imaging device 500. A user output device 564, also located outside the patient, comprises a display portion for displaying the images transmitted by the positionable imaging device 500 and may comprise some status information, for example. A power conditioning module 566, located outside the patient, accepts facility power or batteries and modifies it to suit the energy requirements of the various subsystems, including the energy requirements of the positionable imaging device 500. The power conditioning module 566 may include circuitry to convert energy to frequencies suitable for wireless energy transmission, as indicated by arrow 572, through the skin of the patient, and possibly between the first and second body portions 502a, b of the positionable imaging device 500.

Electric power may undergo many conversions in level and frequency throughout the system 550. For example, 120 VAC, 60 Hz, may enter the system 550. This voltage may be stepped down to 5 VDC, 3.3 VDC, and 1.8 VDC, for example, for use inside the external module 554. Energy may be transferred through the skin of the patient by driving a primary coil with several tens of volts and a few hundred kHz on the outside and coupling some of the energy with a secondary coil located within the first body portion 502a attached to the peritoneal wall 418. A different frequency and voltage may be generated in the first body portion 502a for transferring signals and power over the shorter distance to the second body portion 502b. Alternatively, in one embodiment, wires may be employed to carry the energy to the positionable imaging device 500. Such wires may be introduced inside the patient transcutaneously or translumenally to couple to the fixed first body portion 502a and the movable second body portion 502b.

An orientation management module 568, may be located within the first or second body portions 502a, b to position the positionable imaging device 500 in a desired line of sight. The orientation management module 568 comprises, for example, the necessary circuitry to control the electromagnetic elements 512_{1-q} and may further comprise various feedback devices to stabilize and maintain the desired orientation of the second body portion 502b relative to the first body portion 502a, and hence, the imaging device 508. The orientation management module 568 also includes any necessary mechanical structures necessary for positioning the imaging device 508 such as hinges and/or springs used to interconnect the two body portions 502a, b.

In one embodiment, an angle drive control module may be employed to control the relative angle θ between the first and second body portions 502a, b of the positionable imaging device 500. In one embodiment, the angle drive control module may function in open loop mode with no feedback, the angle-controlling motors (magnets) being activated with a signal derived from the user input device 562. In another embodiment, the relative angle θ, rates or accelerations may be measured (optically, by magnetic sensing, by accelerometers) and incorporated in a control (servo) loop to follow the operator's command from the user input device 562. It will be appreciated by those skilled in the art that a control loop may be required if the individually controllable electromagnetic elements 512_{1-q} are used to manipulate the movable second body portion 502b of the positionable imaging device 500. In one embodiment, the control loop may incorporate sensors in the fixed first body portion 502a as well. Either or both sets of sensors may be used not only to follow the operator's commands from the user input device 562, but to stabilize the orientation (at least the angular orientation) against patient movement that may result from breathing, digestive peristalsis, or circulatory pulsation. The sensors may respond to relative position or movement between the first and second body portions 502a, b, absolute motion (inertial), gravity-sensing, or a combination thereof.

In one embodiment the individually controllable electromagnetic elements 512_{1-q} may be located in the fixed first body portion 502a and permanent magnets may be located in the movable second body portion 502b rather than the individually controllable electromagnetic elements 512₁₋ᵣ discussed with reference to FIGS. 8, 9A, 9B. Furthermore, as in common induction motors, it may be possible to induce a sufficient magnetic field in pole-pieces located in the movable second body portion 502b to allow motion without actually using either electromagnets or permanent magnets located within the movable second body portion 502b.

In one embodiment, the movable second body portion 502b may be provided with a low friction roll axis. Under the influence of the individually controllable electromagnetic elements 512_{1-q} (e.g., spin coils) located in the fixed first body portion 502a, the movable second body portion 502b can be made to spin at high frequency to form a gyroscopic rotor. Synchronous forcing individually controllable electromagnetic elements 512_{1-q} in the fixed first body portion 502a may be pulsed to apply a torque to the rotor, causing it to precess to a new angular orientation. In the absence of forces, the movable second body portion 502b maintains its line of sight, which would be advantageous for clinical applications. The imaging device 508 may be fixed to the pivot axis to prevent it from rotating. In another embodiment, the imaging device 508 may be offset from the pivot point to form a circular scan of the field of view.

In various embodiments, the movable second body portion 502b also may be manipulated by pull cables, pushrods, stacked wedge segments, or other miniature drive components. Such purely mechanical approaches may be alternatives to or supplement the magnetic drive, for example.

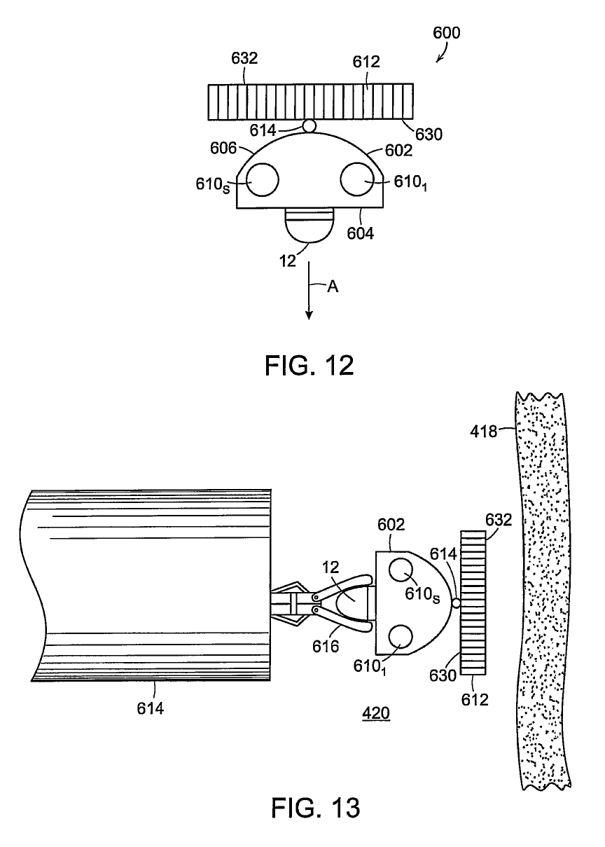

FIG. 12 illustrates one embodiment of a positionable imaging device 600. In one embodiment, the positionable imaging device 600 comprises a body 602 defining a first end 604 and a second end 606. The body 602 is configured to be received within an internal body cavity 420 (FIG. 13) of a patient such as the peritoneal cavity. The body 602 may be shaped according to specific positioning and imaging requirements and, in the illustrated embodiment the body 602 has a substantially hemispherical, ellipsoid shaped dome or rounded configuration. One or more magnetic elements 610₁₋ₛ, where s is any positive integer, is located on the body 602. A first side 630 of a base portion 612 is coupled to the body 602 via a pivotable attachment joint 614 and a second side 632 of the base portion 612 is configured to attach to the internal body wall 418, e.g., the peritoneal wall. As shown in FIG. 13, the positionable imaging device 600 may be deployed via a flexible endoscope 614 with a grasper 616 for holding the positionable imaging device 600 during deployment.

In one embodiment, the positionable imaging device 600 comprises one embodiment of the imaging device 10 described in FIG. 1 for viewing inside body cavities and for transmitting at least video data. The positionable imaging device 600 may be employed for viewing inside body cavities in direction “A” through the optical window 12 located at the first end 604 of the body 602. The optical window 12 may have a hemispherical, ellipsoid shaped dome or rounded configuration. When the positionable imaging device 600 is deployed within the internal body cavity 420 (FIG. 13) of the patient and attached to the patient's anatomy, e.g., the internal body wall 418, the imaging system 10 can acquire images in direction “A.”

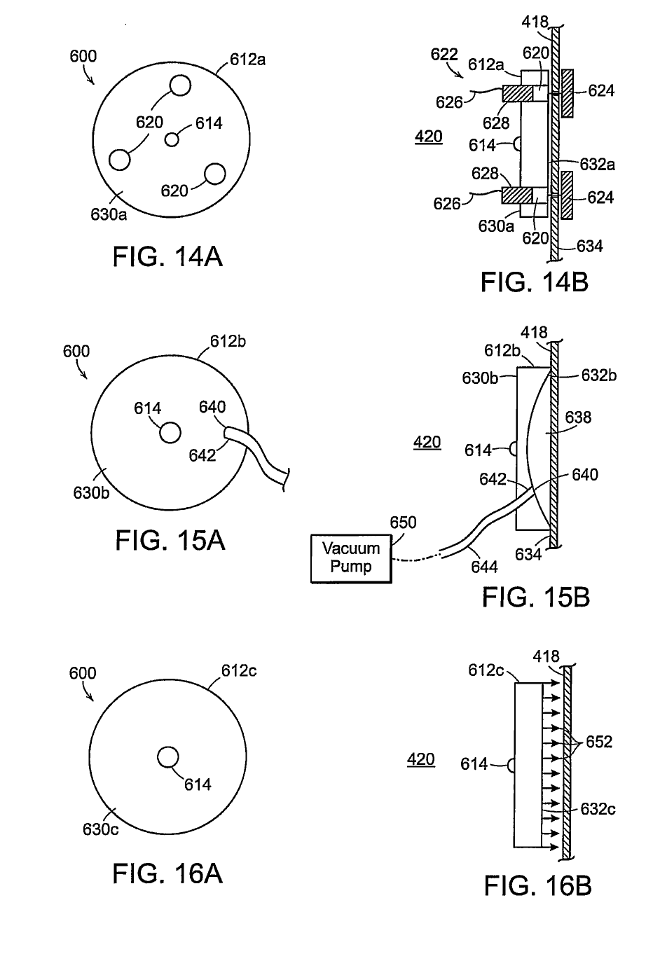

As shown in FIGS. 14A, 14B, 15A, 15B, 16A, and 16B, the base portion 612 of the positionable imaging device 600 is configured to releasably attach to tissue within the internal body cavity 420 such as the internal body wall 418 (FIGS. 14-16). In the embodiment illustrated in FIGS. 14A, 14B, a plurality of openings 620 are formed in a base portion 612a to receive a tissue fastener 622 therethrough. In the illustrated embodiment, the tissue fastener 622 comprises a tissue anchor 624, a length of suture 626, and a knotting element 628. The tissue anchor 624 may be a T-tag, which may be applied using a T-tag tissue apposition system (TAS), for example. The tissue anchor 624 may be inserted in each one of the plurality of openings 620 and is penetrated through the internal body wall 418. The knotting element 626 is formed or applied at a first side 630a of the base portion 612a and the length of suture 626 is tensioned until a second side 632a of the base portion 612a is in contact with the internal portion 634 of the internal body wall 418. When the base portion 612a is attached to the internal body wall 418 the imaging device 10 (FIGS. 12, 13) faces internally towards the peritoneal cavity 420. The base portion 612a may be released from the internal body wall 418 by severing the sutures 626 or releasing the knotting element 626.

In the embodiment illustrated in FIGS. 15A, 15B, a vacuum chamber 638 is formed in a base portion 612b and a fluid port 640 is in fluid communication with the vacuum chamber 638. A first end 642 of a fluid line 644 is fluidically coupled to the fluid port 640 such that the fluid line 644 is in fluid communication with the vacuum chamber 638. A second end of the fluid line 648 is fluidically coupled to a vacuum pump 650. In use, a second side 632b of the base portion 612b is placed in contact with the internal portion 634 of the internal body wall 418. A vacuum is then applied by the vacuum pump 650 to the vacuum chamber 638 to attach the base portion 612b to the internal body wall 418. When the base portion 612b is attached to the internal body wall 418 the imaging device 10 (FIGS. 12, 13) faces internally towards the peritoneal cavity 420. The base portion 612b may be released from the internal body wall 418 be releasing, e.g., venting, the vacuum in the vacuum chamber 638.

In the embodiment illustrated in FIGS. 16A, 16B, a plurality of barbs 652 to penetrate internal tissue such as the internal body wall 418 are formed on a second side 632c of a base portion 612c. When the base portion 612c is attached to the internal body wall 418 the imaging device 10 (FIGS. 12, 13) faces internally towards the peritoneal cavity 420. The base portion 612c may be removed from the internal body wall 418 by applying a pulling force on the positionable imaging device 600 using the graspers 616 (FIG. 13), for example.

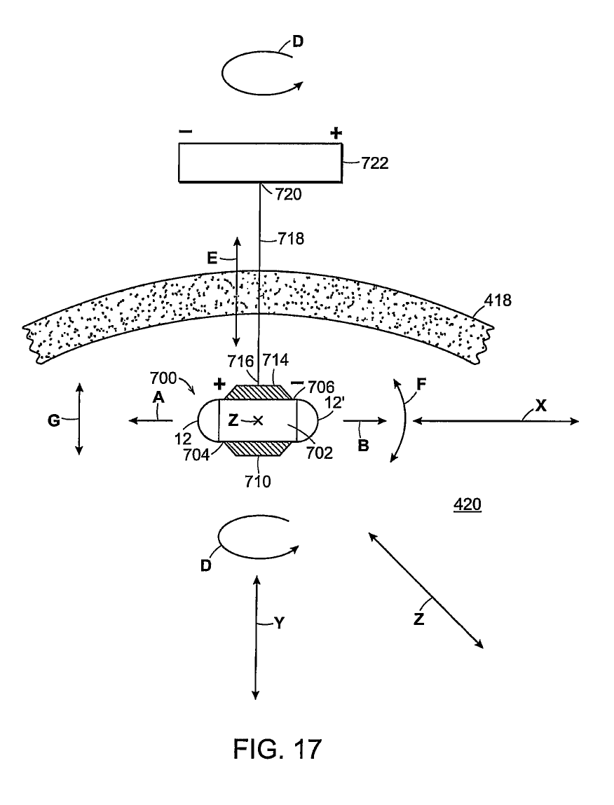

FIG. 17 illustrates one embodiment of a positionable imaging device 700. In one embodiment, the positionable imaging device 700 comprises a body 702 defining a first end 704 and a second end 706. The body 702 is configured to be received within an internal body cavity 420 of a patient such as the peritoneal cavity. A magnetic element 710 is located on the body 702. The positionable imaging device 700 may be deployed via a flexible endoscope 614 with a grasper 616 for holding the positionable imaging device 700 during deployment as previously discussed with respect to FIG. 13 or through conventional open surgical techniques.

In one embodiment, the positionable imaging device 700 comprises one embodiment of the imaging device 10 described in FIG. 1 for viewing inside body cavities and for transmitting at least video data. The positionable imaging device 700 may be employed for viewing inside body cavities in direction “A” through the optical window 12 located at the first end 704 of the body 702. In one embodiment, the positionable imaging device 700 may comprise another optical window 12′ located at the second end 706 of the body 702 for viewing inside body cavities in direction “B.” The first and second optical windows 12, 12′ each may have a hemispherical, ellipsoid shaped dome or rounded configuration. In various embodiments, the positionable imaging device 700 may comprise one or more imaging devices 10 and optical windows 12, 12′ such that the viewing direction “A” or “B” may be selectable by the user. Thus, when the positionable imaging device 700 is deployed within the internal body cavity of the patient and attached to the patient's anatomy, the imaging system 10 can acquire images in either/or both direction “A” or “B.”

In one embodiment, the magnetic element 710 comprises a cylindrical magnet 714 fixedly attached to the body 702. In other embodiments, the magnetic element 710 may comprise a bar magnet or a solid cylindrical magnet may be disposed on the body 702. The cylindrical magnet 714 having a first pole at the first end 704 of the body 702 and second pole at the second end 706 of the body 702. A first end 716 of a suspensory percutaneous filament 718 is fixedly attached to the cylindrical magnet 714 at a substantially intermediate point between the first and second ends 704, 706 of the body 702 such that the cylindrical magnet 714 and the body 702 are substantially balanced. A second end 720 of the suspensory percutaneous filament 718 is attached to a control magnet 722 located outside the internal body wall 418. The control magnet 722 is oriented such that the polarity of the control magnet 722 is opposite that of the cylindrical magnet 714. In the illustrated embodiment, the positionable imaging device 700 is shown in use with the second end 720 of the suspensory percutaneous filament 718 attached to the control magnet 722.

In the illustrated embodiment, the control magnet 722 is located outside the internal body wall 418 such that a clinician can manipulate the positionable imaging device 700 using a combination of the control magnet 722 and the suspensory percutaneous filament 718. Manipulation of the control magnet 722 causes the cylindrical magnet 714 to move correspondingly. The movement can be angular, rotational, or linear. The positionable imaging device 700 can be moved angularly in direction “G” about a first axis X, rotationally in direction “D” about a second axis Y, and angularly in direction “F” about a third axis Z, and linearly “E” along the second axis Y. The rotational movement “D” can be achieved by rotating the control magnet 722. The angular movements “F” and “G” can be achieved by tilting the control magnet with respect to the first, second, and third axes X, Y, Z to achieve a desired orientation. Linear movement “E” can be achieved by pulling or pushing the suspensory percutaneous filament 718 in a corresponding direction. Thus, the body 702 of the positionable imaging device 700 can be independently manipulated and remotely oriented and rotated to position either the first or second optical windows 12, 12′ at a desired viewing orientation to capture a desired field of view of the anatomy within the internal body cavity 420, e.g., the peritoneal cavity, from outside the patient to visualize the desired anatomy. In the illustrated embodiment, the positionable imaging device 700 provides an intra-peritoneal view of the anatomy.

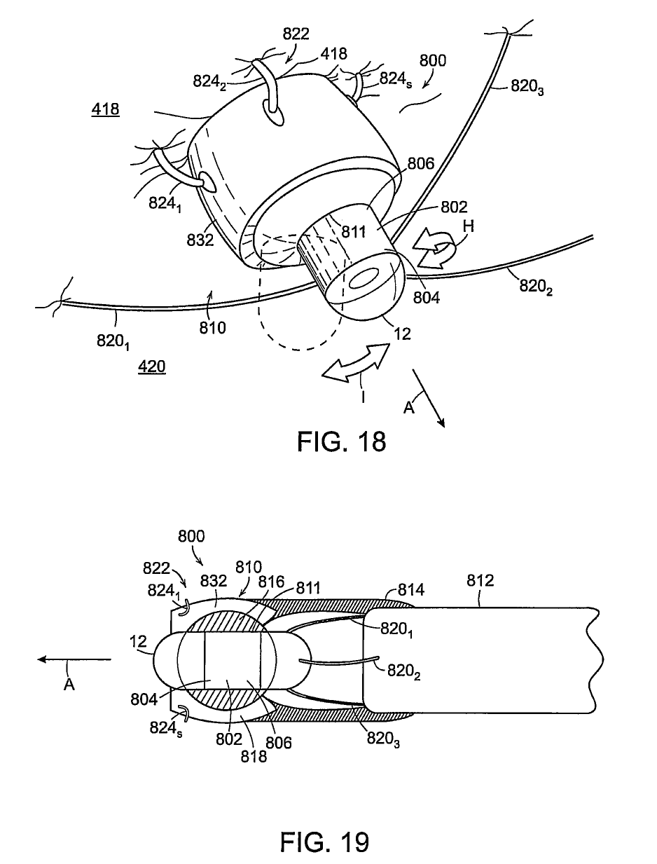

FIG. 18 illustrates a perspective view of one embodiment of a positionable imaging device 800 attached to an internal body wall 418 with one or more fasteners 822. In one embodiment, the positionable imaging device 800 comprises a body 802 defining a first end 804 and a second end 806. The body 802 may be shaped according to specific positioning and imaging requirements and, in the illustrated embodiment the body 802 has a substantially cylindrical configuration. A movable joint 810 comprising a ball 816 and socket 818 arrangement (FIGS. 19-22) is capable of motion around an indefinite number of axes, which have one common center. The ball 816 portion of the movable joint 810 is adapted for fixedly receiving the body 802 within an opening 811 defined therethrough. As illustrated more clearly in FIGS. 19-22, the body 802 is configured to be fixedly engage the inner walls defined by the opening 811 formed through the ball 816 portion of the movable joint 810. The entire assembly comprising the body 802 and the movable joint 810 are configured to be received within an internal body cavity of a patient such as the peritoneal cavity 420. The positionable imaging device 800 may be deployed via a deployment mechanism, which may be introduced into the patient via a flexible endoscope.

The one or more fasteners 822 are rotatably deployable and are rotatably attached to the socket 818 to removably attach the positionable imaging device 800 to the tissue of the internal body wall 418 within the internal body cavity such as the peritoneal cavity 420. In one embodiment, the fasteners 822 comprise a plurality of deployable hooks 824₁₋ₛ, where s is any suitable positive integer. When the positionable imaging device 800 is delivered to the peritoneal cavity 420, the hooks 824₁₋ₛ are deployed to attach a base portion 832 of the movable joint 810 to the internal body wall 418. It will be appreciated that other fasteners 822 may be employed to attach the base portion 832 of the movable joint 810 to the internal body wall 418. As previously discussed, these other attachment mechanism may include, for example, tissue anchors or fasteners, sutures, vacuum devices, and/or barbs. The positionable imaging device 800 is capable of motion around an indefinite number of axes, which have one common center. At least one percutaneous filament 820 (e.g., percutaneous filaments 820_{1, 2, 3}) is attached to the body 802 to manipulate the body 802 of the positionable imaging device 800 in directions indicated by arrows “H” and “I,” and any combination thereof. The embodiments, however, are not limited in this context.

In one embodiment, the positionable imaging device 800 comprises one embodiment of the imaging device 10 described in FIG. 1 for viewing inside body cavities and for transmitting at least video data. The imaging device 10 is located at the first end 804 of the body 802. The positionable imaging device 800 may be employed for viewing inside body cavities in direction “A” through the optical window 12 located at the first end 804 of the body 802. The optical window 12 may have a hemispherical, ellipsoid shaped dome or rounded configuration. Thus, when the positionable imaging device 800 is deployed within the internal body cavity 420 such as the peritoneal cavity and attached to the internal body wall 418, e.g., the peritoneal wall, the imaging system 10 can acquire images in either/or both direction “A.”

FIG. 19 is a partial cross-sectional view of one embodiment of the positionable imaging device 800 coupled to a deployment mechanism 812. As shown in FIG. 19, the ball 816 defines an opening 811 therethrough to fixedly engage the cylindrical portion of the body 802. The socket 818 defines a cup-like portion to rotatably engage the ball 816 such that the ball 816 is capable of freely rotating within the socket 818 without translating within the socket 818. Thus, the body 802 is capable of rotating about a number of axes, which have one common center. The socket 818 portion also serves as the base 832 of the positionable imaging device 800 and comprises the deployable attachment mechanism 822, which in the illustrated embodiment is implemented as the hooks 824₁₋ₛ.

The positionable imaging device 800 may be deployed via the deployment mechanism 812, which may be introduced into the patient using endoscopic, laparoscopic, or open surgical techniques. The deployment mechanism 812 comprises a grasper 814 for holding the positionable imaging device 800 during the deployment stage.

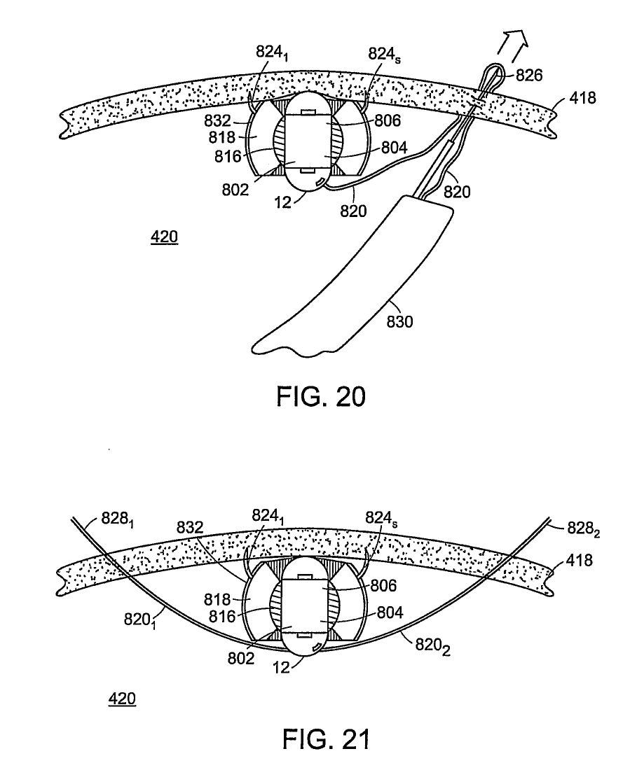

FIG. 20 is a partial cross-sectional view of one embodiment of the positionable imaging device 800 shown in the deployed stage attached to the internal body wall 418 by the fasteners 822. As shown in FIG. 20, the positionable imaging device 800 is attached to the internal body wall 418 by way of the plurality of deployable hooks 824₁₋ₛ. When the base portion 832 of the movable joint 810 is attached to the internal body wall 418, a needle 826 is advanced through a working channel of a flexible endoscope 830 to insert the percutaneous filaments 820₁₋₃ through the internal body wall 418 to locate the ends of the percutaneous filaments 820₁₋₃ outside the internal body wall 418 (FIG. 21). The process is repeated until each of the percutaneous filaments 820₁₋₃ is inserted through the internal body wall 418 and the ends of the percutaneous filaments 820₁₋₃ are located outside the internal body wall 418.

FIG. 21 illustrates a partial cross-sectional view of one embodiment of the positionable imaging device 800 attached to the internal body wall 418 with the one or more hooks 824₁₋ₛ. The ends 828_{1, 2} of the respective percutaneous filaments 820₁₋₂ protruding through the internal body wall 418 are used to manipulate the body 802 of the positionable imaging device 800 from outside the patient.

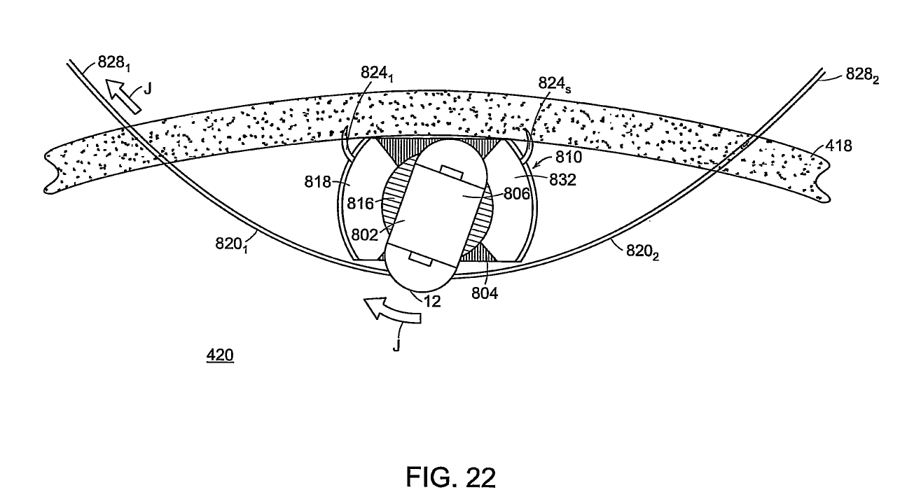

FIG. 22 shows the body 802 rotatably positioned as a result of applying a force in direction “J” on the end 828₁ of the respective percutaneous filament 820₁ from outside the internal body wall 418, e.g., from outside the body of the patient. The body 802 of the positionable imaging device 800 can be manipulated by manipulating the ends 828_{1, 2} of the percutaneous filaments 820_{1, 2}. Accordingly, the imaging device 10 of the positionable imaging device 800 can be rotated by tugging on the respective ends 828_{1, 2} of the percutaneous filaments 820_{1, 2} from outside the internal body wall 418. For example, when the percutaneous filaments 820₁ is pulled in direction “J,” the body 802 of the positionable imaging device 800 rotates within the socket 818 portion of the movable joint 810 in direction “J.” The ball 816 and socket 818 arrangement enables the body 802 of the positionable imaging device 800 to freely rotate to obtain a desired view of the peritoneal cavity 420.

In various embodiments, any one of the positionable imaging devices 100, 200, 300, 400, 500, 600, 700, and 800 disclosed herein may be introduced into the patient using a variety of endoscopic, laparoscopic, or conventional laparotomy techniques. Endoscopic techniques include minimally invasive techniques to access the internal anatomy of a patient or NOTES™ techniques where an imaging device may be inserted into the patient through a natural opening such as the mouth, vagina, or anus. Laparoscopic techniques enable access to the internal anatomy of a patient through small incisions or keyholes penetrating the abdominal wall to reach the peritoneal cavity. Laparoscopic techniques are usually performed using trocars. Conventional laparotomy techniques include access techniques where open incisions are made through the abdominal wall to access to the peritoneal cavity. In one embodiment, an imaging device may be configured to be ingested by the patient and advanced through the alimentary canal through a process known as peristalsis. The embodiments are not limited in this context.

Prior to intubating any one of the positionable imaging devices 100, 200, 300, 400, 500, 600, 700, and 800 disclosed herein into an endoscopic trocar, the endoscopist (e.g., clinician, physician, or surgeon) may insert the positionable imaging device into an applier. The positionable imaging device and applier assembly then may be introduced through a flexible endoscopic trocar and may be deployed at the desired anatomical location (e.g., worksite or deployment site) or internal body cavity such as the peritoneal cavity using an integral attachment mechanism. Any one of the positionable imaging devices 100, 200, 300, 400, 500, 600, 700, and 800 described herein may be deployed in a desired tissue plane using the integral attachment mechanism. The embodiments, however, are not limited in this context as other techniques may be employed to deliver the camera to the target worksite.

In one embodiment, the applier may be suitably configured to releasably engage any one of the positionable imaging devices 100, 200, 300, 400, 500, 600, 700, and 800 disclosed herein and to couple to a deployment handle via a shaft. The shaft may be flexible and suitable for deploying the applier and the camera via an inner working channel of a flexible endoscope, for example. The deployment handle may be coupled to the camera via the applier through the shaft. In flexible endoscopic translumenal procedures, a flexible/articulating shaft enables the applier to traverse the tortuous paths of the natural openings of the patient through the working channel of a flexible endoscope. For example, the shaft can me made suitably flexible or may comprise articulated elements to make it suitable to traverse the GI tract.

As previously discussed, the attachment mechanism may comprise one or more fasteners. In the illustrated embodiment, the fasteners are formed as needle-like hooks suitable for penetrating tissue and attaching the positionable imaging device thereto. The attachment mechanism may be actuated by engaging features formed on the body of the positionable imaging device using commercially available instruments or the applier. The applier may be configured to deploy, position, reposition, or remove any one of the positionable imaging devices 100, 200, 300, 400, 500, 600, 700, and 800 disclosed herein. The deployment handle may comprise deployment and reversing triggers to deploy and remove the attachment mechanism when the camera is attached at the desired position. A description of one example of a deployment handle and applier for a imaging device mechanism is provided in commonly owned U.S. patent application Ser. No. 12/170,862, titled “Temporarily Positionable Medical Devices” and U.S. patent application Ser. No. 11/166,610, now United States Patent Application Publication No. US 2005/0283118, titled “Implantable Medical Device With Simultaneous Attachment Mechanism And Method,” each of which is incorporated herein by reference. The embodiments, however, are not limited in this context.

Any one of the features of the positionable imaging devices 100, 200, 300, 400, 500, 600, 700, and 800 described with respect with one embodiment may be readily substituted and combined with features of other embodiments without limitation.

Any one of the positionable imaging devices 100, 200, 300, 400, 500, 600, 700, and 800 disclosed herein may be employed during natural orifice translumenal endoscopic procedures to provide images of the surgical site that are similar in quality and orientation to those obtainable in open or laparoscopic procedures. For example, in laparoscopic procedures, a laparoscope may be rotated about its optical axis, translated forward and rearward, and may be rotated about a pivot point defined by a trocar or tissue keyhole site to control its orientation and obtain a quality image at a desired viewing angle. During laparoscopic procedures, a clinician can manipulate the laparoscope to provide an optimal image of the surgical site. In addition, the laparoscope can be used to pan and/or zoom the images while the clinician manipulates the laparoscope independently of manipulating tissue or organs proximate to the surgical site.

The positionable imaging devices 100, 200, 300, 400, 500, 600, 700, and 800 disclosed herein can be designed to be disposed of after a single use, or they can be designed to be used multiple times. In either case, however, the positionable devices can be reconditioned for reuse after at least one use. Reconditioning can include any combination of the steps of disassembly of the positionable device, followed by cleaning or replacement of particular pieces, and subsequent reassembly. In particular, the positionable device can be disassembled, and any number of the particular pieces or parts of the positionable device can be selectively replaced or removed in any combination. Upon cleaning and/or replacement of particular parts, the positionable device can be reassembled for subsequent use either at a reconditioning facility, or by a surgical team immediately prior to a surgical procedure. Those skilled in the art will appreciate that reconditioning of a positionable device can utilize a variety of techniques for disassembly, cleaning/replacement, and reassembly. Use of such techniques, and the resulting reconditioned positionable device, are all within the scope of the present application.

Preferably, the various embodiments described herein will be processed before surgery. First, a new or used positionable device is obtained and if necessary cleaned. The positionable device can then be sterilized. In one sterilization technique, the positionable device is placed in a closed and sealed container, such as a plastic or TYVEK® bag. The container and the positionable device are then placed in a field of radiation that can penetrate the container, such as x-rays, or high-energy electrons. The radiation kills bacteria on the instrument and in the container. Other sterilization techniques, such as Ethylene Oxide (EtO) gas sterilization also may be employed to sterilize the positionable device prior to use. The sterilized positionable device can then be stored in the sterile container. The sealed container keeps the positionable device sterile until it is opened in the medical facility.

It is preferred that the positionable device is sterilized. This can be done by any number of ways known to those skilled in the art including beta or gamma radiation, ethylene oxide, steam.

Although various embodiments have been described herein, many modifications and variations to those embodiments may be implemented. For example, different types of end effectors may be employed. Also, where materials are disclosed for certain components, other materials may be used. The foregoing description and following claims are intended to cover all such modification and variations.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

In summary, numerous benefits have been described which result from employing the concepts described herein. The foregoing description of the one or more embodiments has been presented for purposes of illustration and description. It is not intended to be exhaustive or limiting to the precise form disclosed. Modifications or variations are possible in light of the above teachings. The one or more embodiments were chosen and described in order to illustrate principles and practical application to thereby enable one of ordinary skill in the art to utilize the various embodiments and with various modifications as are suited to the particular use contemplated. It is intended that the claims submitted herewith define the overall scope.

What is claimed is:

1. A positionable imaging device, comprising:
a body defining a first end and a second end, the body configured to be received within an internal body cavity;
an imaging device located at the first end of the body;
a releasable fastener coupled to the body to removably attach the imaging device to tissue within the internal body cavity; and
a release mechanism coupled to the releasable fastener to detach the imaging device from the tissue.

2. The positionable imaging device of claim 1, comprising an elongate memory alloy having a first end and second end, the first end is attached to the body, wherein the memory alloy is actuatable from a first state to a second state by an energy source coupled between the first and second ends of the memory alloy.

3. The positionable imaging device of claim 2, comprising:
a pin coupled to the first end of the memory alloy to removably couple the memory alloy to the body; and
a filament having a first end and a second end, the first end coupled to a tissue anchor and the second end defining a loop removably coupled to the pin;
wherein when the memory alloy is actuated, the memory alloy transitions from the first state to the second state and the pin is slidably released from the loop to disconnect the pin from the body.

4. The positionable imaging device of claim 3, comprising first and second axially aligned projections formed on the body defining corresponding first and second openings to slidably receive the pin.

5. The positionable imaging device of claim 2, comprising a remotely actuatable switch coupled between the first end of the memory alloy and the energy source.

6. A positionable imaging device, comprising:
a body defining a first end and a second end, the body configured to be received within an internal body cavity;
an imaging device located at the first end of the body; and
a plurality of percutaneous filaments each having a first and second end, the first end fixedly attached to the body.

7. The positionable imaging device of claim 6, comprising a collar fixedly attached to the body, wherein the first end of each of the plurality of percutaneous filaments is fixedly attached to the collar.

8. A positionable imaging device, comprising:
a body defining a first end and a second end, the body configured to be received within an internal body cavity;
an imaging device located at the first end of the body; and
a magnetic element fixedly attached to the body.

9. The positionable imaging device of claim 8, comprising a magnetic interface positioned outside the internal body cavity to coact with the magnetic element to remotely position the imaging device at a desired viewing angle from outside the body cavity.

10. The positionable imaging device of claim 8, comprising a magnetic collar circumferentially positioned over the body and fixedly attached thereto.

11. The positionable imaging device of claim 10, wherein the magnetic collar is configured to coact with a control rod located outside the patient to remotely position the imaging device at a desired viewing angle and rotate the imaging device from outside the patient by manipulating the control rod.

12. The positionable imaging device of claim 8, comprising an electromagnet comprising a plurality of electromagnetic elements configured to coact with the magnetic element to remotely position the imaging device at a desired viewing angle and to rotate the imaging device from outside the patient by selectively energizing one or more of the plurality of electromagnetic elements.

13. The positionable imaging device of claim 8, comprising:
a first body portion configured to attach to internal tissue, the first body portion comprising a first plurality of individually controllable electromagnetic elements arranged in a predetermined pattern; and
a second body portion coupled to the first body portion by a coupling member, the second body portion comprising a second plurality of individually controllable electromagnetic elements arranged in the predetermined pattern and the second body portion movable relative to the first body portion in response to the first and second plurality of individually controllable electromagnetic elements, the second body portion comprising the imaging device;
wherein the first and second plurality of electromagnetic elements are remotely coupled to a controller to remotely position the imaging device at a desired viewing angle and to rotate the second body portion from outside the patient by selectively energizing one or more of the first and second plurality of individually controllable electromagnetic elements.

14. The positionable imaging device of claim 13, wherein the first plurality of individually controllable electromagnetic elements arranged in a first circular array and wherein the second plurality of individually controllable electromagnetic elements arranged in a second circular array.

15. The positionable imaging device of claim 13, comprising:
a first circuit coupled to the first body portion to control the activation of the first plurality of individually controllable electromagnetic elements; and
a second circuit coupled to the second body portion to control the activation of the second plurality of individually controllable electromagnetic elements.

16. The positionable imaging device of claim 13, comprising a plurality of fasteners located on the first body portion to removably attach the first body portion to tissue within the internal body cavity.

17. The positionable imaging device of claim 8, comprising a base portion pivotally attached the body, the base portion configured to removably attach to tissue within the internal body cavity.

18. The positionable imaging device of claim 17, comprising a plurality of openings formed in the base portion to receive a tissue fastener therethrough.

19. The positionable imaging device of claim 17, comprising:
a vacuum chamber formed in the base portion; and
a fluid port in fluid communication with the vacuum chamber.

20. The positionable imaging device of claim 17, comprising a plurality of barbs to penetrate the tissue within the internal body cavity.

21. The positionable imaging device of claim 8, comprising:
a cylindrical magnet fixedly attached to the body; and
a percutaneous filament having a first end and a second end, the first end fixedly attached to the cylindrical magnet the second end configured to attach to a control magnet.

22. The positionable imaging device of claim 28, comprising a control magnet attached to the second end of the percutaneous filament.

23. A positionable imaging device, comprising:
a body defining a first end and a second end, the body configured to be received within an internal body cavity;
an imaging device located at the first end of the body; and
a movable joint that is capable of motion around an indefinite number of axes, which have one common center, the movable joint defining an opening to fixedly receive the body therein.

24. The positionable imaging device of claim 23, wherein the movable joint comprises a ball and socket arrangement.

25. The positionable imaging device of claim 24, wherein the ball defines the opening configured to fixedly receive the body therein to hold the body and to enable the body to rotate about a number of axes.

26. The positionable imaging device of claim 23, comprising at least one percutaneous filament comprising a first end attached to the second end of the body.

27. The positionable imaging device of claim 26, comprising a plurality of fasteners rotatably attached to the movable joint to removably attach the tissue within the wherein the at least one percutaneous filament is attached to the body to manipulate the body of the imaging device in various directions.
